(19) United States
(12) Patent Application Publication
(10) Pub. No.: US 2016/0243040 A1
(43) Pub. Date: Aug. 25, 2016

(54) Pharmaceutical Composition with Antiinflammatory Agents and Production Process

(71) Applicant: LABORATORIO RAAM DE SAHUAYO, S.A. DE C.V., Sahuayo (MX)

(72) Inventors: Federico Amezcua Amezcua, Sahuayo (MX); Amador Covarrubias Pinedo, Zapopan (MX)

(21) Appl. No.: 14/909,509

(22) PCT Filed: Jul. 24, 2014
(86) PCT No.: PCT/MX2014/000117

§ 371 Date: May 10, 2016

(30) Foreign Application Priority Data
Aug. 2, 2013 (MX) MX/A/2013/008992

(51) Int. Cl. A61K 9/24; A61K 31/216; A61K 9/20; A61K 31/573

(52) U.S. Cl. CPC A61K 9/209; A61K 31/573; A61K 31/216; A61K 9/2095

(57) ABSTRACT

The present invention relates to pharmaceutical compositions including a triphasic release system, which may be delayed release and/or extended release and/or modified release and/or immediate release, of at least three layers for the formation of at least one dosage unit. Each layer includes, as active pharmaceutical ingredients, at least one corticosteroid agent of the betamethasone type and/or the pharmaceutically acceptable salts thereof, at least one non-steroidal anti-inflammatory agent of the Aceclofenac type and/or the pharmaceutically acceptable salts thereof, and at least one pharmaceutically acceptable excipient. Also described is the novel production process. Said triphasic release system generates an enhanced treatment effect to combat inflammation and body pain.

### FIELD OF THE INVENTION

The present invention relates to novel pharmaceutical compositions comprising a three-phase release system having at least three layers to form at least one oral dosage unit, consisting of at least two outer layers, with at least one core spatially located, the layers may have a delayed-release system, and/or an extended-release system, and/or a controlled-release system, and/or an immediate-release system containing at least one corticosteroid agent, and at least one non-steroidal anti-inflammatory, and at least one pharmaceutically acceptable excipient, a novel production process and method of manufacture thereof, useful for reducing inflammation and treating body pain.

### BACKGROUND

The adrenal glands are multifunctional endocrine organs secreting various steroids, which can be divided into corticosteroids based in a 21-carbon core and sex steroids, mainly androgens, based on a 19-carbon core. Corticosteroids are traditionally divided by their metabolic activities into those with glucocorticoid actions, of which cortisol (hydrocortisone) is the most important endogenous example, and those with electrolyte regulation, which primarily are mineralocorticoids, of which aldosterone is the most important. Glucocorticoids are the ones who have more therapeutic applications. (Sweetman, S. 2002)

Numerous experimental and clinical observations have shown that the adrenal glands are essential for life. Their secretions play a wide variety of physiological functions such as regulation of blood glucose, protein turnover, metabolism of fat, balance of sodium, potassium and calcium, modulation of tissue response to injuries or infections and, above all, survival to any stress. (Berne, L. 1998)

Glucocorticoids are potent suppressors of inflammation. These can prevent or suppress inflammation in response to multiple provoking phenomena, including radiant, mechanical, chemical, infectious, and immune stimuli. Although the use of glucocorticoids as anti-inflammatory does not attack the root cause of the disease, the suppression of inflammation has enormous clinical utility, and has led these compounds to be among those most frequently prescribed. Similarly, glucocorticoids are immensely useful for treating diseases originated by undesirable immune reactions, including diseases that occur predominantly by humoral immunity, like hives. (Goodman and Gilman. 2003)

The effects of corticosteroids include: abnormal metabolism of carbohydrates, proteins and lipids; conservation of fluid and electrolyte balance, and preservation of normal function of the cardiovascular and immune systems, kidneys and striated muscle, as well as the endocrine and nervous systems. In addition, corticosteroids allow the body to withstand circumstances that generate stress, as harmful stimuli and environmental changes. Corticoids also have an effect at the level of absorption and excretion of calcium, resulting in a decrease in reserves of calcium in the body. (Goodman and Gilman, Sweetman, S. 2003, 2002)

Corticosteroids are grouped according to their relative potencies of retaining sodium, actions on carbohydrate metabolism (i.e., glycogen deposition in the liver and glycogenesis) and anti-inflammatory effects as shown in Table 1.

TABLE 1
Relative potencies and equivalent doses
of representative corticosteroids.

| | Anti-inflam- | Potency to | Duration | Dose equiv- |
|---|---|---|---|---|
| Compound | matory potency | retain Na+ | of action | alents ♦mg |
| Cortisol | 1 | 1 | B | 20 |
| Cortisone | 0.8 | 0.8 | B | 25 |
| Fludrocortisone | 10 | 125 | I | ** |
| Prednisone | 4 | 0.8 | I | 5 |
| Prednisolone | 4 | 0.8 | I | 5 |
| 6α-methyl | 5 | 0.5 | I | 4 |
| prednisolone | | | | |
| Tiamcinolone | 5 | 0 | I | 4 |
| Betamethasone | 25 | 0 | P | 0.75 |
| Dexamethasone | 25 | 0 | P | 0.75 |

B, short (Biological half-life of 8-12 h);
I, intermediate (Biological half-life of 12-36 h);
P, prolonged (Biological half-life of 36 to 72 h).
♦These dose relationships apply only to the oral or intravenous use, since the powers of glucocorticoids may differ long after intramuscular or intra-articular administration.
** This compound is not used for obtaining glucocorticoid effects.

The anti-inflammatory and immunosuppressive properties of glucocorticoids make them invaluable therapeutic agents in numerous diseases, but do not attack the underlying cause of the disease.

Glucocorticoids influence the traffic of circulating leukocytes and immune accessory cells. They suppress immune activation of these cells, inhibit the production of cytokines and other mediators of inflammation, and produce resistance to cytokines. Reduce or prevent tissue response to inflammatory processes, thereby reducing the development of inflammation symptoms without affecting the underlying cause. These drugs inhibit the accumulation of inflammatory cells, including leukocytes and macrophages. They also inhibit phagocytosis, the release of lysosomal enzyme, and the synthesis and/or release of chemical mediators of inflammation.

Hydrocortisone and many congeners, including synthetic analogs, are effective when administered orally. Most of the corticosteroids that are used systemically are hydroxy (alcohol) compounds. They are relatively insoluble in water and the sodium salt of phosphate or succinate ester is generally used to provide water-soluble forms for solutions. These esters are hydrolyzed easily in the body.

Betamethasone has a very important anti-inflammatory property; it intervenes on phospholipase A2 by blocking the production of arachidonic acid, avoiding the cascade of chemical mediators involved in the inflammatory process.

Concomitant use of glucocorticoids with non-steroidal anti-inflammatory drugs (NSAIDs) can provide an additive therapeutic effect, which allows to reduce the dose of glucocorticoids.

Non-steroidal anti-inflammatory drugs (NSAIDs) are a group of drugs that have anti-inflammatory, analgesic and antipyretic action, which act by inhibiting the activity of cyclooxygenase enzymes that convert arachidonic acid found in cell membranes in unstable cyclic endoperoxydes that are transformed into prostaglandins (PGs) and thromboxanes, which are involved in the pathogenic mechanisms of inflammation, pain and fever (Feria, 1997).

Aceclofenac is a non-steroidal anti-inflammatory drug (NSAID), derived from aryl acetic acid, structurally related to diclofenac. It is a potent inhibitor of the cyclooxygenase enzyme, which is involved in the production of prostaglandins.

The pharmacokinetic properties of aceclofenac have been studied after oral administration of single or multiple doses in healthy young and elderly volunteers, as well as in patients with osteoarthritis.

Following oral administration of a dose of 100 mg to healthy young volunteers, maximum plasma concentration (Cmax) of aceclofenac at 6.8 to 8.9 mg/L was reached between 1.4 to 2 hours (Tmax). The duration of treatment had only a small effect on the pharmacokinetic parameters of aceclofenac, which were similar after a single dose or multiple doses in young or elderly volunteers. The presence of food did not modify the pharmacokinetics of aceclofenac, except tmax, from 2.0 to 3.5 hours.

The average aceclofenac concentration in synovial fluid reached half that of plasma after treatment with 225 mg of aceclofenac daily for 6 days.

It is metabolized mainly into 4-hydroxi-aceclofenac; the other metabolites, diclofenac and 4-hydroxy-diclofenac, only represent 5% of an administered dose.

Approximately 70% of the dose is excreted in urine, mainly as aceclofenac glucuronide and diclofenac, and its hydroxy metabolites; the rest is excreted in feces. After a single dose of 100 mg aceclofenac, the elimination half-life is 6.2 hours.

Concomitant administration of an oral single dose of glucocorticoid and a non-steroidal anti-inflammatory is essential for the treatment and/or control of inflammation and/or body aches and/or diseases related thereto.

The main applications are: For the treatment of acute pain associated with headache, dental procedures, minor surgery, with oral administration, rapid absorption and short half-life. Also chronic pain, fever, osteoarthritis, rheumatoid arthritis, gout, primary dysmenorrhea, cardiovascular disease, Alzheimer's disease, tendonitis, myalgia, neuralgia, acute rheumatic fever, diseases associated with platelet aggregation (coronary artery disease, acute myocardial infarction, transient cerebral ischemia, etc.), among other diseases.

In the state of the art are described patents protecting a kit and method of administration of aceclofenac esters and betamethasone esters in aerosol (U.S. Pat. No. 7,078,019); a method of manufacturing of a solid dispersion comprising dissolve a selected water-insoluble drug of aceclofenac and betamethasone and a substitute of cyclodextrin in an organic solvent and drying under reduced pressure these dispersions to improve the speed of dissolution of drugs and maximize its bioavailability (W003043602); a therapeutic composition and treatment methods, containing betamethasone, aceclofenac, an immunosuppressant, and an IRM component (W02005055932); a pharmaceutical composition containing betamethasone, aceclofenac and other compounds to inhibit COX and LOX, its process of preparation and method of use against inflammation and/or pain, and/or disorders associated (W02007072503); a pharmaceutical composition containing azelastine or betamethasone and aceclofenac for transnasal or ocular administration (W02006058022); a device for administering aceclofenac or betamethasone to respiratory system.

Body pain with inflammation of the affected areas is a very common public health problem, which over time can become a risk factor that may lead to a poor quality of life and restrict physical activity in general. For this purpose, an improvement and/or control of such ailments can be achieved by means of a treatment, managing to have a significant impact at the population level.

### OBJECT OF THE INVENTION

The present invention relates to novel pharmaceutical compositions with a three-phase release system, consisting of at least three layers, with at least one spatially located core, which may have a delayed-release system, and/or a prolonged-release system and/or a controlled-release system, and/or an immediate-release system, where each layer may contain at least one corticosteroid agent and/or at least one non-steroidal anti-inflammatory agent, and at least one pharmaceutically acceptable excipient, wherein said active pharmaceutical ingredients provides synergistic clinical effects at the time they are released from the formulation in order to achieve optimized therapeutic effects. A novel production process of solid oral dosages for the treatment and/or control of body pain, and/or local, and/or general inflammation in the body is also presented.

### DETAILED DESCRIPTION OF THE INVENTION

The present invention relates to novel solid pharmaceutical compositions obtained using a new production process, comprising a three-phase release system capable of reducing side effects and/or dependency from treatment, among some other benefits, when they are in the same composition and different from those obtained by the independent administration of pharmaceutically active agents.

The administration of these novel solid pharmaceutical compositions, which have a three-phase release system, allow to have benefits such as increased efficiency of therapeutic effect and/or pharmaceutically active agents in lower concentrations and/or lower dose, and/or more extended times for administration and/or lower risk of some liver damage.

Such solid pharmaceutical compositions are composed of at least three layers to form an oral dosage unit, wherein at least each layer contains at least one pharmaceutically active agent belonging to group a) corticosteroid and at least one agent belonging to group b) non-steroidal anti-inflammatory drug, plus one or more pharmaceutically acceptable excipients. The pharmaceutically active agents, when administered orally in a single dosage form concomitantly cause a better therapeutic effect.

The present invention is characterized by comprising a novel system of distribution of pharmaceutically active agents contained in an oral dosage unit with a three-phase release system. The release system is tree-phase because the oral dosage unit is comprised of at least three layers, wherein the first layer consists of a core located in the center of the oral dose unit; the second underlayer covers at least half of the central core; the third upper layer completely covers the central core and gives the final form of the oral dosage unit.

According to the above, the pharmaceutically active agent of the corticosteroid group, and/or salts, and pharmaceutically acceptable excipients thereof are present in the first layer; the pharmaceutically active agent of the non-steroidal anti-inflammatory group, and/or their salts and pharmaceutically acceptable excipients are present in the second and third layers.

The three-phase release system of the solid dosage unit was determined from the behavior and/or pharmacokinetic characteristics of these pharmaceutically active agents when administered in an oral dosage; also, the times for release of each pharmaceutically active agent are adjusted primarily to reduce side effects that they can cause and/or adverse reactions associated with the administration thereof, in order to obtain an effective, efficient and safe pharmaceutical composition with respect to those reported in the state of the art.

These tablets that form the oral dosage unit may have a delayed release system, and/or an extended release system, and/or a modified release system, and/or an immediate-release system.

The three-phase release system for pharmaceutically active agents of the dosage unit is useful for the treatment and/or control of body pain and/or inflammation of the same and/or related diseases.

The three-phase release system contains the synergistic combination of such pharmaceutically active agents contained in at least one solid dosage unit for oral administration composed of at least three layers as follows: the first layer or core comprising at least a corticosteroid or pharmaceutically acceptable salts thereof with a delayed-release system, and/or an extended-release system, and at least one pharmaceutically acceptable excipient; a second underlayer comprising at least one non-steroidal anti-inflammatory and/or pharmaceutically acceptable salts thereof with a modified-release system, and/or an immediate-release system, and at least one pharmaceutically acceptable excipient; a third top layer comprising at least one non-steroidal anti-inflammatory, and/or pharmaceutically acceptable salts thereof with an extended-release system, and at least one pharmaceutically acceptable excipient. Preferably, for the object of the present invention is used at least one core containing a corticosteroid agent and/or pharmaceutically acceptable salts thereof having a delayed release system; at least one underlayer containing a non-steroidal anti-inflammatory agent and/or pharmaceutically acceptable salts thereof, having an immediate-release system and at least one top layer containing a non-steroidal anti-inflammatory agent and/or pharmaceutically acceptable salts thereof having an extended release system, in order to avoid antagonistic competition for cytochrome P-450 in the liver, allowing to have the synergistic effect of the two pharmaceutically active agents.

Novel compositions product of this invention are characterized because the core comprises a) at least one pharmaceutically active corticosteroid and/or its pharmaceutically acceptable salts, i) at least one diluent and/or ii) at least one binding agent, and/or iii) at least one release polymer, and/or iv) at least one lubricant and/or vii) at least one solvent; the underlayer comprises b) at least one pharmaceutically active non-steroidal anti-inflammatory agent and/or its pharmaceutically acceptable salts or i) at least one diluent, and/or ii) at least one binding agent and/or iii *) at least one disintegrating agent, and/or iv) at least one lubricant, and/or v) at least one solvent; the top layer comprises c) at least one pharmaceutically active non-steroidal anti-inflammatory agent or its pharmaceutically acceptable salts and/or at least one diluent or at least one binding agent and/or at least one release polymer, and/or at least one lubricant, and/or at least one solvent.

In addition, the layers forming the oral dosage unit may or may not contain other pharmaceutically acceptable excipients selected from: pH modifiers and/or alkalizing agents, among others.

The pharmaceutically active corticosteroid a) that is present in the core is present in a concentration ranging from 0.10 to 10 mg and is selected from the following group: hydrocortisone, cortisone, corticosterone, prednisone, prednisolone, 6α-methyl-prednisolone, tiamcinolone, betamethasone, dexamethasone, as well as their pharmaceutically acceptable salts and/or polymorphs and/or prodrugs and/or metabolites and/or amorphous and/or hemisolvates of each; and/or combinations thereof.

The pharmaceutically active group belonging to non-steroidal anti-inflammatory agent b) that is present in the underlayer is present in a concentration ranging from 10 to 125 mg and is selected from the following group: aceclofenac, acetylsalicylic acid, ketoprofen, etodolac, flurbiprofen, piroxicam, indomethacin, ibuprofen, sulindac, naproxen, diclofenac, fenoprofen and their pharmaceutically acceptable salts, and/or polymorphs, and/or prodrugs, and/or metabolites, and/or amorphous, and/or hemisolvates of each of them; and/or combinations thereof.

The pharmaceutically active agent of the group belonging to non-steroid anti-inflammatory agents c) that is present in the top layer is present in a concentration range that goes from 10 to 125 mg and is selected from the following group: aceclofenac, acetylsalicylic acid, ketoprofen, etodolac, flurbiprofen, piroxicam, indomethacin, ibuprofen, sulindac, naproxen, diclofenac, fenoprofen, and their pharmaceutically acceptable salts, and/or polymorphs, and/or prodrugs, and/or metabolites, and/or amorphous, and/or hemisolvates from each of them; and/or combinations thereof.

The diluent i) for each layer is selected from: starch, and/or pre-gelatinized starch, and/or corn starch, and/or sugar, and/or sugar compressible, and/or isomalt, and/or microcrystalline cellulose, and/or lactose, and/or sorbitol, and/or sucrose, and/or tragacanth, and/or talc, and/or trehalose, and/or xylitol, and/or the combination of at least two of the above, among others.

The binder agent ii) for each layer is from: acacia, and/or agar, and/or alginic acid, and/or calcium carbonate, and/or calcium lactate, and/or carbomer, and/or carboxymethyl cellulose calcium, and/or microcrystalline cellulose, and/or cellulose, and/or ceratonia, and/or chitosan, and/or copovidone, and/or dextrates, and/or dextrin, and/or dextrose, and/or ethyl cellulose, and/or gelatin, and/or glyceryl behenate, and/or guar gum, and/or hydroxyethyl cellulose, and/or hydroxymethyl cellulose, and/or low-substituted hydroxypropyl cellulose, and/or hydroxypropyl starch, and/or methylcellulose, and/or inulin, and/or lactose monohydrate, and/or magnesium aluminum silicate, and/or maltodextrin, and/or methylcellulose, and/or polycarbophil, and/or polydextrose, and/or polyethylene oxide, and/or polymethacrylates, and/or polyvinylpyrrolidone, and/or iodine, and/or sodium alginate, and/or starch, and/or pre-gelatinized starch, and/or sucrose starch, and/or polyethylene glycol succinate, and/or zein, and/or the combination of at least two of the above, among others.

The release polymer agent iii) for the core and the top layer is selected from: acetiltributil citrate, and/or acetyl trietil citrate, and/or calcium carbonate, and/or carboxymethyl cellulose calcium and/or carboxymetyl cellulose sodium, and/or cellulose acetate, and/or cellulose, and/or ceresin, and/or cetyl alcohol, and/or chitosan, and/or diethyl phthalate, and/or dimethyl phthalate, and/or ethylcellulose, and/or gelatin, and/or liquid glucose, and/or glycerin, and/or glyceryl behenate, and/or glyceryl palmitostearate, and/or hydroxyethyl cellulose, and/or hydroxymethyl cellulose, and/or hydroxypropyl cellulose, and/or hypromellose, and/or hypromellose succinate, and/or hypromellose phthalate, and/or isomaltitol, and/or maltitol, and/or maltodextrin, and/or methylcellulose, and/or paraffin, and/or poloxamer, and/or polydextrose, and/or poly-LD-lactic acid, and/or polyethylene oxide, and/or polymethacrylates, and/or poli(metil vinil ether/maleic anhydride), and/or polyvinyl acetate phthalate, and/or shellac, and/or sodium chloride, and/or stearic acid, and/or sucrose, and/or talc, and/or titanium dioxide, and/or triacetin, and/or tributyl citrate, and/or triethyl citrate, and/or triolein, and/or carnauba wax, and/or microcrystalline wax, and/or white wax, and/or yellow wax, and/or xylitol, and/or zein, and/or carbomer and their derivatives, and/or the combination of at least two of the above, among others.

The disintegrating agent iii *) for the underlayer is selected from the following group: alginic acid, and/or crospovidone, and/or ion exchange resins, and/or aluminum silicate, and/or magnesium silicate, and/or microcrystalline cellulose, and/or starch, and/or sodium glycolate starch, and/or modified cellulose gum, and/or PVP, and/or sodium dodecyl sulphate, and/or corn starch, and/or rice starch, and/or cross-linked N-vinyl-2-pyrrolidone, and/or croscarmellose sodium, and/or formaldehyde-casein, and/or a combination of at least two of the above, among others.

The lubricant iv) is selected from: calcium stearate, and/or glyceryl behenate, and/or glyceryl monostearate, and/or glyceryl palmitostearate, and/or lauric acid, and/or leucine, and/or magnesium stearate, and/or maltodextrin, and/or mineral oil, and/or light mineral oil, and/or myristic acid, and/or palmitic acid, and/or polyethylene glycol, and/or polyvinyl alcohol, and/or potassium benzoate, and/or sodium chloride, and/or sodium hyaluronate, and/or sodium lauryl sulfate, and/or sodium stearyl fumarate, and/or stearic acid, and/or talc, and/or hydrogenated vegetable oil, and/or zinc stearate, and/or a combination of at least two of the above, among others.

The solvent agent v) for each layer is selected from acetone, and/or methanol, and/or ethanol, and/or absolute ethanol, and/or 80% ethanol, and/or 90% ethanol, and/or chloroform, and/or benzyl alcohol, and/or benzyl benzoate, and/or butylene glycol, and/or castor oil, and/or corn oil, and/or cottonseed oil, and/or dibutyl phthalate, and/or diethyl phthalate, and/or dimethyl phthalate, and/or dimethyl sulfoxide, and/or dimethylacetamide, and/or ethyl acetate, and/or ethyl lactate, and/or ethyl oleate, and/or glycerine, and/or glycofurol, and/or isopropyl alcohol, and/or isopropyl myristate, and/or isopropyl palmitate, and/or chain triglycerides medium, and/or mineral oil, and/or light mineral oil, and/or octyldodecanol, and/or peanut oil, and/or polyethylene glycol, and/or propylene carbonate, and/or propylene, and/or pyrrolidone, and/or safflower, and/or sesame oil, and/or soybean oil, and/or sunflower oil, and/or triacetin, and/or tricaprylin, and/or triethyl citrate, and/or triolein, and/or water, and/or a combination of at least two of these, among others.

The agent for pH modification is selected from: adipic acid, and/or boric acid, and/or calcium carbonate, and/or calcium lactate, and/or meglumine, and/or calcium phosphate, and/or citric acid monohydrate, and/or glycine, and/or maleic acid, and/or methionine, and/or monosodium glutamate, and/or potassium citrate, and/or sodium acetate, and/or sodium borate, and/or sodium carbonate, and/or sodium citrate dihydrate, and/or sodium hydroxide, and/or dibasic sodium phosphate, and/or monobasic sodium phosphate, and/or a combination of at least two of these, among others.

The alkalizing agent is selected from the following group: potassium citrate, and/or sodium citrate, and/or sodium hydroxide, and/or potassium hydroxide, and/or sodium bicarbonate, and/or sodium carbonate, and/or calcium bicarbonate, and/or calcium carbonate, and/or potassium bicarbonate, and/or disodium phosphate, and/or trisodium phosphate, and/or arginine, and/or talc, and/or 1-leucine, and/or the combination of at least two of these, among others.

Examples of novel pharmaceutical compositions containing the concomitant combination of a pharmaceutically active corticosteroid and a pharmaceutically active non-steroidal anti-inflammatory, as well as suitable excipients are also described; which present a novel three-phase release system. However, the scope of the present invention is not limited to these examples:

### EXAMPLE 1

### Pharmaceutical Composition with Three-Phase Release System

| | |
|---|---|
| Component | Concentration (mg) |

Core (delayed release)

| | |
|---|---|
| Betamethasone | 0.10-10 |
| Microcrystalline cellulose | 2.5-4 0 |
| Lactose | 2.5-40 |
| Methylcellulose | 10-95 |
| Polyvinyl pyrrolidone | 1-15 |
| Magnesium stearate | 1-10 |
| Ethanol | 0.5-4 |

Underlayer (immediate release)

| | |
|---|---|
| Aceclofenac | 10-125 |
| Microcrystalline cellulose | 10-100 |
| Lactose | 10-100 |
| Cornstarch | 10-100 |
| Polyvinyl pyrrolidone | 1-50 |
| Crospovidone | 1-50 |
| Stearic acid | 1-15 |
| Ethanol | 0.5-4 |

Top layer (extended release)

| | |
|---|---|
| Aceclofenac | 10-125 |
| Microcrystalline cellulose | 10-100 |
| Lactose | 10-100 |
| Cornstarch | 10-100 |
| Polyvinyl pyrrolidone | 1-50 |
| Methylcellulose | 10-95 |
| Stearic acid | 1-15 |
| Ethanol | 0.5-4 |

### EXAMPLE 2

### Pharmaceutical Composition with Three-Phase Release System

| | |
|---|---|
| Component | Concentration (mg) |

Core (extended release)

| | |
|---|---|
| Betamethasone | 0.10-10 |
| Microcrystalline cellulose | 2.5-40 |
| Lactose | 2.5-40 |
| Pre-gelatinized starch | 2.5-40 |
| Hydroxypropyl methyl cellulose | 10-95 |
| Talc | 1-15 |
| Calcium stearate | 1-10 |
| Absolute ethanol | 0.5-4 |

Underlayer (immediate release)

| | |
|---|---|
| Aceclofenac | 10-125 |
| Microcrystalline cellulose | 10-100 |
| Lactose | 10-100 |
| Pre-gelatinized starch | 10-100 |
| Polyvinyl pyrrolidone | 1-50 |
| Crospovidone | 1-50 |
| Calcium stearate | 1-15 |
| Absolute ethanol | 0.5-4 |

Top layer (delayed release)

| | |
|---|---|
| Aceclofenac | 10-125 |
| Microcrystalline cellulose | 10-100 |
| Lactose | 10-100 |
| Pre-gelatinized starch | 10-100 |
| Talc | 1-50 |
| Hydroxypropyl methyl cellulose | 10-95 |
| Calcium stearate | 1-15 |
| Absolute ethanol | 0.5-4 |

Furthermore, such pharmaceutical compositions with three-phase release system are characterized by having a humidity of between 2.5% and 5% in powder; a hardness of between 6 kg and 9 kg; and friability less than 1.

Process for preparing the solid oral dosage unit with three-phase release system:

For the core:

1. Mix the pharmaceutically active corticosteroid, one or more diluents and one or more binders.

2. Granulate the above mixture with one or more solvents, sieve, dry, and sieve through a mesh.

3. Add and mix one or more polymeric release agents.

4. Add and mix one or more lubricants.

5. Compress.

For the underlayer:

1. Mix the pharmaceutically active non-steroidal anti-inflammatory, one or more diluents, and one or more binders.

2. Granulate the above mixture with one or more solvents, sieve, dry, and sieve through a mesh.

3. Add and mix one or more disintegrating agents.

4. Add and mix one or more lubricants.

For the top layer:

1. Mix the pharmaceutically active non-steroidal anti-inflammatory, one or more diluents, and one or more binders.

2. Granulate the above mixture with one or more solvents, sieve, dry, and sieve through a mesh.

3. Add and mix one or more polymeric release agents.

4. Add and mix one or more lubricants.

Tableting:

1. Dose in each of the hoppers the core, the powder for the underlayer and the powder for the top layer.

2. Compress the powder and core with a proper punch.

Importantly, this pharmaceutical composition with a three-phase release system offers in a non-limitative way, advantages such as: an effective, efficient and safe concomitant pharmacological effect; doses of the pharmaceutically active agents are low, which prevents secondary and/or side effects to the body by oral administration of both.

1. A pharmaceutical composition, comprising a three-phase release system with at least one corticosteroid agent and/or at least one non-steroidal anti-inflammatory agent and at least one pharmaceutically acceptable carrier, useful for the treatment of body pain and/or inflammation of affected areas.

2. The pharmaceutical composition according to claim 1, wherein the pharmaceutically active agent selected from the group of corticosteroids is hydrocortisone, and/or cortisone, and/or corticosterone, and/or prednisone, and/or prednisolone, and/or 6a-methyl-prednisolone, and/or tiamcinolone, and/or betamethasone, and/or dexamethasone, and/or its pharmaceutically acceptable salts, and/or polymorphs, and/or prodrugs, and/or metabolites, and/or amorphous, and/or hemisolvates of each; and/or combinations thereof.

3. The pharmaceutical composition according to claim 1, wherein the pharmaceutically active agent selected from the group of non-steroidal anti-inflammatory agents is aceclofenac, and/or acetylsalicylic acid, and/or ketoprofen, and/or etodolac, and/or flurbiprofen, and/or piroxicam, and/or indomethacin, and/or ibuprofen, and/or sulindac, and/or naproxen, and/or diclofenac, and/or fenoprofen, and/or pharmaceutically acceptable salts, and/or polymorphs, and/or prodrugs, and/or metabolites, and/or amorphous, and/or hemisolvates of each, and/or combinations thereof.

4. The pharmaceutical composition according to claim 1, wherein the pharmaceutically acceptable corticosteroid is betamethasone, as well as pharmaceutically acceptable salts, and/or polymorphs, and/or prodrugs, and/or metabolites, and/or amorphous, and/or hemisolvates thereof.

5. The pharmaceutical composition according to claim 1, wherein the pharmaceutically acceptable non-steroidal anti-inflammatory agent is aceclofenac, as well as pharmaceutically acceptable salts, and/or polymorphs, and/or prodrugs, and/or metabolites, and/or amorphous, and/or hemisolvates thereof.

6. The pharmaceutical composition according to claim 1, wherein the pharmaceutically active corticosteroid agent is betamethasone and is in a concentration of 0.1 to 10 mg, and the pharmaceutically active non-steroidal anti-inflammatory agent is aceclofenac and is in a concentration of 10 to 125 mg.

7. The pharmaceutical composition according to claim 1, wherein a dosage unit is in the form of a three-phase tablet, salve, and/or ointment, patch, topical gel, intramuscular injectable, topical solution, and/or spray.

8. The pharmaceutical composition according to claim 7, wherein at least one oral dosage unit is made.

9. The pharmaceutical composition according to claim 8, wherein the oral dosage unit is a tablet.

10. The pharmaceutical composition according to claim 9, wherein the tablet consists of three layers and has a three-phase release system.

11. The pharmaceutical composition according to claim 9, wherein the tablet as a whole has a three-phase release system.

12. The pharmaceutical composition according to claim 10, wherein the three-phase release system is comprised of at least one spatially located core, an underlayer, and a top layer.

13. The pharmaceutical composition according to claim 12, wherein the core has a delayed-release system and/or an extended-release system; the underlayer has an immediate-release system and/or a modified-release system; the top layer has an extended- and/or delayed-release system.

14. The pharmaceutical composition according to claim 13, wherein the core has a delayed-release system; the lower layer has an immediate-release system; and the top layer has an extended-release system.

15. The pharmaceutical composition according to claim 12, wherein the core comprises one or more excipients and/or pharmaceutically acceptable additives selected from at least one diluent, and/or at least one binder, and/or at least one release polymer, and/or at least one lubricant, and/or at least one solvent, and/or at least one pH modifier, and/or at least one alkalizing agent.

16. The pharmaceutical composition according to claim 12, wherein the underlayer comprises one or more excipients and/or pharmaceutically acceptable additives selected from at least one diluent, and/or at least one binder, and/or at least one disintegrant, and/or at least one lubricant, and/or at least one solvent, and/or at least one pH modifier, and/or at least one alkalizing.

17. The pharmaceutical composition according to claim 12, wherein the underlayer comprises one or more excipients and/or pharmaceutically acceptable additives selected from at least one diluent, and/or at least one binder, and/or at least one release polymer, and/or at least one lubricant, and/or at least one solvent, and/or at least one pH modifier, and/or at least one alkalizing agent.

18. A process for the preparation of a pharmaceutical composition according to claim 1, wherein the process comprises the following production steps:
a) for a core:
i) mix the pharmaceutically active corticosteroid, one or more diluents and one or more binders;
ii) granulate the above mixture with one or more solvents, sieve, dry, and sieve through a mesh;
iii) add and mix one or more polymeric release agents;
iv) add and mix one or more lubricants;
v) compress the powder with a suitable punch;
b) for an underlayer:
i) mix the pharmaceutically active non-steroidal anti-inflammatory, one or more diluents, and one or more binders;
ii) granulate the above mixture with one or more solvents, sieve, dry, and sieve through a mesh;
iii) add and mix one or more disintegrating agents;
iv) add and mix one or more lubricants;
c) for a top layer:
i) mix the pharmaceutically active non-steroidal anti-inflammatory, one or more diluents, and one or more binders;
ii) granulate the above mixture with one or more solvents, sieve, dry, and sieve through a mesh;
iii) add and mix one or more polymeric release agents;
iv) add and mix one or more lubricants;
d) for tableting of core (a), and powders, (b) and (c):
i) dose in each of the hoppers the core, the powder for the underlayer and the powder for the top layer;
ii) compress the powder and core with a suitable punch.
